(19) United States
(12) Patent Application Publication
(10) Pub. No.: US 2026/0287594 A1
(43) Pub. Date: Sep. 24, 2026

(54) DIAGNOSIS METHOD USING CELL FREE NUCLEOSOME LEVELS

(71) Applicant: Belgian Volition SRL, Isnes (BE)

(72) Inventors: Andrew RETTER, Isnes (BE); Jacob Vincent MICALLEF, Isnes (BE); Remi RABEUF, Isnes (BE)

(21) Appl. No.: 19/474,679

(22) PCT Filed: Apr. 15, 2024
(86) PCT No.: PCT/EP2024/060181

§ 371 Date: Oct. 10, 2025

(30) Foreign Application Priority Data
Apr. 14, 2023 (GB) 2305531.2
Apr. 14, 2023 (GB) 2305534.6
Apr. 14, 2023 (GB) 2305537.9
May 15, 2023 (GB) 2307208.5

(51) Int. Cl. G01N 33/68; G01N 33/86

(52) U.S. Cl. CPC G01N 33/6875; G01N 33/6854; G01N 33/6893; G01N 33/86; G01N 2800/226; G01N 2800/50; G01N 2800/52

(57) ABSTRACT

The invention relates to methods of detecting a subject in need of medical treatment for a disorder of hemostasis or thrombosis by detecting the level of cell free nucleosomes or a component thereof.

### FIELD OF THE INVENTION

The present invention relates to the use of cell free nucleosomes as biomarkers in body fluid samples for patients with a disorder of hemostasis or thrombosis, and also for identifying patients at high risk of developing a disorder of hemostasis or thrombosis.

### BACKGROUND OF THE INVENTION

Problems with the body's ability to control how the blood clots (coagulate) can lead to hemostasis and thrombosis disorders. Conditions falling under this definition include, but are not limited to, venous thromboembolism, vasculitis, disseminated intravascular coagulopathy and antiphospholipid syndrome.

Venous thromboembolism (VTE) is the third most common cause of cardiovascular illness and is projected to double in incidence by 2050. It is a spectrum of disease that includes deep vein thrombosis (DVT) and pulmonary embolism (PE). VTE is a diagnostically challenging illness that can cause significant disability and death if not promptly diagnosed and effectively treated. Acute PE is a common and often fatal disease with a mortality rate of around 30% without treatment. While mortality can be reduced by prompt diagnosis and therapy, it is estimated that more than half of all patients with PE remain undiagnosed. The magnitude of VTE as a clinical problem can be attributed in part due to the wide variety of patient presentations, and limited diagnostic and therapeutic options.

The D-dimer test is currently used to assist in the diagnosis of VTE in clinical practice. This test measures the dimeric forms of the fibrin degradation products using an antigen-antibody reaction. One known problem with the D-dimer test in humans is poor specificity and poor positive predictive value of a positive D-dimer test.

Imaging examinations such as ultrasound imaging for the diagnosis of DVT and scintigraphy or angiography to diagnose PE. These imaging methods are expensive, can carry significant morbidity and hence are deployed late in the diagnosis process. Moreover, depending upon the clinical setting it may not be possible to access such imaging methods within a therapeutically useful timeframe. Since the disease process is so variable from asymptomatic to life threatening, prompt and accurate diagnosis is vital and can improve mortality significantly.

Additionally the conventional diagnostic approach is inefficient as it is unable to exclude VTE in high-risk patients. Moreover, the optimal strategy for diagnosing recurrent VTE remains unclear.

Vasculitis is the name for a group of conditions that cause inflammation of the blood vessels. Inflammation is the immune system's natural response to injury or infection. It causes swelling and can help the body deal with invading germs. However, in vasculitis the immune system attacks healthy blood vessels, causing them to become swollen and narrow. This may be triggered by an infection, another underlying condition, or a medicine, although often the cause is unknown. Vasculitis can range from a minor problem that just affects the skin, to a more serious problem that causes problems with organs like the heart or kidneys.

Since complete remission from some forms of vasculitis is achievable in case of early diagnosis, there is a need for diagnostic markers of vasculitis, in particular, of markers allowing early diagnosis of condition.

Disseminated intravascular coagulopathy (DIC) occurs because of aberrant activation of the clotting cascade, leading to fibrin deposition in small vessels, combined with activation of fibrinolytic mechanisms, leading to bleeding. DIC is usually a common final haemostatic disorder caused by other conditions such as sepsis, pancreatitis, or trauma. As they are consumed by the ongoing prothrombotic and fibrinolytic processes, coagulation proteins and platelets can become depleted, leading to bleeding. Thus, in DIC, haemorrhage and thrombosis can occur simultaneously. DIC can be an acute or a chronic disorder, and the latter is seen mostly in obstetric and oncology patients. Acute DIC is the form most likely to be encountered in a critical care setting.

It will be appreciated that DIC is a serious, potentially life threatening condition. DIC is a common cause of concurrent thrombocytopenia and prolonged clotting times (activated partial thromboplastin time (aPTT) and prothrombin time (PT)) in hospitalised patients. It can also be an independent predictor of mortality. When detected early, DIC may be treatable. However, there is not a standard procedure for diagnosing DIC. DIC may be identified through various tests related to levels of platelets, clotting factors, and other blood components. Therefore there is a real need for diagnostic markers of DIC, in particular, of markers allowing early diagnosis of the disorder, and which can be used either on their own or which aid diagnosis as part of a panel of tests.

Antiphospholipid syndrome (APS), sometimes known as Hughes syndrome, is an autoimmune condition that causes an increased risk of blood clots. People with APS are at greater risk of developing conditions such as deep vein thrombosis (DVT), arterial thrombosis and blood clots in the brain. Pregnant women with APS also have an increased risk of having a miscarriage. APS can also be associated with general symptoms such as tiredness or numbness and tingling in different parts of the body.

In APS, the immune system produces abnormal antibodies called antiphospholipid antibodies. Antiphospholipid antibodies are a heterogeneous family of immunoglobulins that includes, among others, lupus anticoagulants and anticardiolipin antibodies.

The term ‘lupus anticoagulant’ is a misnomer as it is neither only found in lupus, nor is it mainly associated with bleeding. The term LA was first coined to describe the phenomenon of plasma samples from patients with systemic lupus erythematosus (SLE) that failed to clot within an appropriate time. Lupus anticoagulant (LA) is one the of antiphospholipid antibodies, which also include anticardiolipin (aCL) antibody and anti-beta2-glycoprotein (GP) I antibodies. LA are heterogenous autoantibodies, predominantly IgG and IgM isotypes, that specifically target the phospholipid-protein component of the cell membrane. LA interferes and prolongs the clotting process, which is a risk factor for arterial and/or venous thrombosis with complications such as stroke, transient ischemic strokes, acquired thrombophilia, and pregnancy loss. Furthermore, LAs may be transitory in the setting of certain medications or infections and thus have also present in asymptomatic patients. Testing for LAs is essential in patients with hypercoagulable states and APS.

These immunoglobins may develop spontaneously due to medications, infections, or as a consequence of autoimmune diseases such as lupus erythematosus. Infections such as EBV, syphilis, and hepatitis C increase the likelihood of acquired lupus anticoagulant.

Lupus anticoagulant is estimated to be present in 2 to 4% of the general population, but true prevalence is unclear. Estimates are that lupus anticoagulant is present in 15 to 34% of patients with systemic lupus erythematosus. LA Incidence increases with age and female sex. Laboratory detection of LA is challenging due to the heterogenicity of antibodies and epitopes on protein in the phospholipids. Thus, there is no single LA test capable of detecting all LAs. Clinical laboratories have shown variance in sensitivity and specificity. There are many different mechanisms proposed for this variance, including adequate plasma preparation that is not platelet poor and dilutional effects of mixing studies that impact detection of LA. Laboratory diagnosis of LA may be difficult but could allow for the optimal duration of therapy as the risk of occurrence and recurrence of venous/arterial thromboembolism are high. Initial positive testing for LA activity should be repeated after at least 12 weeks to confirm its persistence as it could be falsely positive due to some drugs and infections.

There remains a need in the art to provide simple, rapid, cost-effective methods to identify and prioritize individuals likely to develop a disorder of hemostasis or thrombosis and to monitor treatment and progress of condition.

### SUMMARY OF THE INVENTION

According to one aspect of the invention there is provided a method of detecting a subject in need of medical treatment for a disorder of hemostasis or thrombosis comprising:
(i) contacting a body fluid sample obtained from the subject with a binding agent to detect or measure the level of cell free nucleosomes or a component thereof; and
(ii) using the level of cell free nucleosomes as an indicator that the subject is in need of medical treatment for a disorder of hemostasis or thrombosis.

According to one aspect of the invention, the disorder of hemostasis or thrombosis is selected from VTE (including DVT and/or PE), vasculitis, DIC or APS.

According to one aspect of the invention there is provided a method of detecting a subject in need of medical treatment for deep vein thrombosis (DVT), comprising:
(i) contacting a body fluid sample obtained from the subject with a binding agent to detect or measure the level of cell free nucleosomes or a component thereof; and
(ii) using the level of cell free nucleosomes as an indicator that the subject is in need of medical treatment for DVT.

According to one aspect of the invention there is provided a method of detecting a subject in need of medical treatment for pulmonary embolism (PE), comprising:
(i) contacting a body fluid sample obtained from the subject with a binding agent to detect or measure the level of cell free nucleosomes or a component thereof; and
(ii) using the level of cell free nucleosomes as an indicator that the subject is in need of medical treatment for PE.

According to another aspect of the present invention there is provided a method for diagnosing the presence or the risk of development of a disorder of hemostasis or thrombosis or of monitoring the progress of a disorder of hemostasis or thrombosis in a subject suffering from the condition, comprising:
(i) contacting a body fluid sample obtained from the subject with a binding agent to detect or measure the level of cell free nucleosomes or a component thereof;
(ii) repeating step (i) on one or more occasions; and
(iii) using any changes in the level of cell free nucleosomes or component thereof to monitor the progression of the disorder of hemostasis or thrombosis in the subject.

According to another aspect of the present invention there is provided a method for diagnosing the presence or the risk of development of DVT or of monitoring the progress of DVT in a subject suffering from the condition, comprising:
(i) contacting a body fluid sample obtained from the subject with a binding agent to detect or measure the level of cell free nucleosomes or a component thereof;
(ii) repeating step (i) on one or more occasions; and
(iii) using any changes in the level of cell free nucleosomes or component thereof to monitor the progression of DVT in the subject.

According to another aspect of the present invention there is provided a method for diagnosing the presence or the risk of development of PE or of monitoring the progress of a PE in a subject suffering from the condition, comprising:
(i) contacting a body fluid sample obtained from the subject with a binding agent to detect or measure the level of cell free nucleosomes or a component thereof;
(ii) repeating step (i) on one or more occasions; and
(iii) using any changes in the level of cell free nucleosomes or component thereof to monitor the progression of the PE in the subject.

According to another aspect of the present invention there is provided a method of assigning a risk of an adverse outcome to a subject suffering from a disorder of hemostasis or thrombosis, comprising:
(i) contacting a body fluid sample obtained from the subject with a binding agent to detect or measure the level of cell free nucleosomes or a component thereof; and
(ii) using the level of cell free nucleosomes detected to assign the likelihood of an adverse outcome to said subject,
wherein a subject identified with a high likelihood of an adverse outcome is assigned for medical intervention.

According to another aspect of the present invention there is provided a method of assigning a risk of an adverse outcome to a subject suffering from DVT, comprising:
(i) contacting a body fluid sample obtained from the subject with a binding agent to detect or measure the level of cell free nucleosomes or a component thereof; and
(ii) using the level of cell free nucleosomes detected to assign the likelihood of an adverse outcome to said subject,
wherein a subject identified with a high likelihood of an adverse outcome is assigned for medical intervention.

According to another aspect of the present invention there is provided a method of assigning a risk of an adverse outcome to a subject suffering from PE, comprising:
(i) contacting a body fluid sample obtained from the subject with a binding agent to detect or measure the level of cell free nucleosomes or a component thereof; and
(ii) using the level of cell free nucleosomes detected to assign the likelihood of an adverse outcome to said subject,
wherein a subject identified with a high likelihood of an adverse outcome is assigned for medical intervention.

### BRIEF DESCRIPTION OF FIGURES

FIG. 1. The results of an immunoassay for neutrophil extracellular trap (NET) derived nucleosomes in EDTA plasma and heparin plasma samples taken from 2 healthy volunteers. The EDTA samples contain low levels of NET derived nucleosome material. In contrast, heparin induces NET formation and the heparin plasma samples contain high levels of induced NET derived nucleosomes.

FIG. 2. Bioanalyzer electrophoresis results for NET derived nucleosomes in EDTA plasma and heparin plasma samples taken from 2 healthy volunteers. The EDTA samples contain low levels of both mononucleosomes and NET derived nucleosome material. In contrast, heparin induces NET formation and the heparin plasma samples contain low levels of mononucleosomes (peak at approximately 60 seconds) but high levels of induced NET derived nucleosomes (wide peak at approximately 110 seconds). The narrow peaks at approximately 43 seconds and approximately 110 seconds represent DNA samples added for reference purposes.

FIG. 3. (A) Box plot analysis representations of the circulating H3.1 nucleosomes (ng/ml) in Deep Vein Thrombosis (DVT) patients and control (healthy) samples. The box represents the 25th-75th percentile with median. Median values are 13.40 for Healthy (n=6) and 79.40 for DVT (n=13). (B) Box plot analysis representations of the circulating H3R8Cit nucleosomes (ng/ml) in DVT patients and control (healthy) samples. The box represents the 25th-75th percentile with median. Median values are 11.40 for Healthy (n=6) and 50.10 for DVT (n=13). The whiskers in both plots include data within the range of the 2.5th to 97.5th percentile. *** represent p-value<0.001, calculated using the Mann-Whitney U test.

### DETAILED DESCRIPTION

The present invention relates to the diagnosis of a disorder of hemostasis or thrombosis, which may include venous thromboembolism (hereinafter “VTE”), which includes deep-vein thrombosis (hereinafter “DVT”) and/or pulmonary embolism (hereinafter “PE”), vasculitis, disseminated intravascular coagulation disorder (hereinafter “DIC”), or antiphospholipid syndrome (hereinafter “APS”).

Blood clotting disorders are sometimes called coagulation disorders or thrombophilias. Therefore, the disorder of hemostasis or thrombosis may be a coagulation disorder. Coagulation disorders may be either inherited or acquired (i.e. as the result of another illness or injury). For example, APS and DIC are types of acquired blood clotting disorders. In one embodiment, the invention relates to the diagnosis of a disorder comprising abnormal or dysregulated coagulation (in particular of blood in the veins).

VTE (including DVT and/or PE) occurs in approximately 1 in 1000 persons per year. Prompt diagnosis and initiation of therapy is vital, since untreated PE has a mortality rate of approximately 30%, and nearly 30% of untreated DVTs will result in severe swelling or ulceration of the leg. With prompt diagnosis and treatment, PE- or treatment-related death is less than 1%. Further, accurately ruling out VTE avoids unnecessary treatment with anticoagulation and the attendant risks. DIC is also referred to as consumption coagulopathy or defibrination syndrome. DIC can be defined as an acute or chronic disorder causing thrombosis or haemorrhage, which occurs as a secondary complication of an underlying disease. It is characterised by consumption of coagulation factors caused by intravascular activation of the coagulation sequence, which leads to the formation of thrombi throughout the microcirculation of the body, and secondarily, activation of fibrinolysis. APS is also referred to as Hughes syndrome. Antiphospholipid antibodies are autoantibodies that are directed against phospholipid-binding proteins. APS may be characterised by the presence of antiphospholipid antibodies in the setting or thrombosis and/or pregnancy loss. Early diagnosis of APS can make a big difference to the lives of those with APS. However, making a diagnosis can be difficult due to a lack of clear guidelines.

Nucleosomes are released into the circulation on fragmentation of chromatin on cell death. Many infections, such as viral infections, initiate cell death through a variety of mechanisms (cell binding and entry, endosomal TLR3 activation and gene expression) thereby increasing the number of circulating nucleosomes in the blood (Danthi et al., Annu. Rev. Virol. (2016) 3:533-53). Whilst not wishing to be bound by any theory we believe disorders of hemostasis or thrombosis are associated with NETosis. In NETosis post-translational histone modifications, such as acetylation or hypercitrullination of histones H3 and H4 (Wang Y et al., J. Cell Biol. (2009) 184 (2): 205-213), promote decondensation of chromatin which is released into circulation together as a first line response to infection. However, extracellular nucleosomes and neutrophil extracellular traps (NETs) can cause severe complications if not cleared rapidly. For example, nucleosome binding to the glomerular membrane is associated with kidney damage in lupus (Kalaaji et al., Kidney Int. (2007) 71 (7): 665-672), whilst NETs have been shown to intensify pulmonary injury during viral pneumonia (Ashar et al., Am. J. Pathol. (2018) 188 (1): 135-148). Indeed, host directed NET toxicity is associated with respiratory distress, occlusion of narrow airways, endothelial and epithelial cell damage, inflammatory response and thrombus formation and other pathologies (Marcos et al., Nat. Med. (2010) 16:1018-23; Hoeksema et al., Future Microbiol. (2016) 11:441-53).

The present invention utilises elevated levels of cell free nucleosomes, including NETs, to assist in the diagnosis of a disorder of hemostasis or thrombosis and to predict severity of condition and outcome in the condition.

Therefore, according to one aspect, there is provided a method of detecting a subject in need of medical treatment for a disorder of hemostasis or thrombosis comprising:
(i) contacting a body fluid sample obtained from the subject with a binding agent to detect or measure the level of cell free nucleosomes or a component thereof; and
(ii) using the level of cell free nucleosomes as an indicator that the subject is in need of medical treatment for a disorder of hemostasis or thrombosis.

According to a further aspect of the invention, there is provided a method of diagnosing a disorder of hemostasis or thrombosis in a subject, comprising:
(i) contacting a body fluid sample obtained from the subject with a binding agent to detect or measure the level of cell free nucleosomes or a component thereof; and
(ii) using the level of cell free nucleosomes as an indicator that the subject has a disorder of hemostasis or thrombosis.

In another aspect there is provided a method for diagnosing the presence or the risk of development of a disorder of hemostasis or thrombosis, or of monitoring the progress of a disorder of hemostasis or thrombosis in a subject suffering from the condition, comprising:
(i) contacting a body fluid sample obtained from the subject with a binding agent to detect or measure the level of cell free nucleosomes or a component thereof;
(ii) repeating step (i) on one or more occasions; and
(iii) using any changes in the level of cell free nucleosomes or component thereof to monitor the progression of the disorder of hemostasis or thrombosis in the subject.

In another aspect there is provided a method of assigning a risk of an adverse outcome to a subject suffering from a disorder of hemostasis or thrombosis comprising:
(i) contacting a body fluid sample obtained from the subject with a binding agent to detect or measure the level of cell free nucleosomes or a component thereof; and
(ii) using the level of cell free nucleosomes detected to assign the likelihood of an adverse outcome to said subject,
wherein a subject identified with a high likelihood of an adverse outcome is assigned for medical intervention.

The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller, Methods Mol. Biol. (2007) 361:25-62).

References to “nucleosome” may refer to “cell free nucleosome” when detected in body fluid samples. It will be appreciated that the term cell free nucleosome throughout this document is intended to include any cell free chromatin fragment that includes one or more nucleosomes.

It will be understood that the cell free nucleosome may be detected by binding to a component thereof. The term “component thereof” as used herein refers to a part of the nucleosome, i.e. the whole nucleosome does not need to be detected. The component of the cell free nucleosomes may be selected from the group consisting of: a histone protein (i.e. histone H1, H2A, H2B, H3 or H4), a histone post-translational modification, a histone variant or isoform, a protein bound to the nucleosome (i.e. a nucleosome-protein adduct), a DNA fragment associated with the nucleosome and/or a modified nucleotide associated with the nucleosome. For example, the component thereof may be histone (isoform) H3.1 or histone H1 or DNA.

Methods and uses of the invention may measure the level of (cell free) nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. As described herein, histone proteins form structural units known as nucleosomes which are used to package DNA in eukaryotic cells.

Normal cell turnover in adult humans involves the creation by cell division of a huge number of cells daily and the death of a similar number, mainly by apoptosis. During the process of apoptosis chromatin is broken down into mononucleosomes and oligonucleosomes which are released from the cells. Under normal conditions the levels of circulating nucleosomes found in healthy subjects is reported to be low. Elevated levels are found in subjects with a variety of conditions including many cancers, auto-immune conditions, inflammatory conditions, stroke and myocardial infarction (Holdenrieder & Stieber, Crit. Rev. Clin. Lab. Sci. (2009) 46 (1): 1-24).

Previous nucleosome ELISA methods were used primarily in cell culture, usually as a method to detect apoptosis (Salgame et al., Nucleic Acids Res. (1997) 25 (3): 680-681; Holdenrieder et al. (2001) supra; van Nieuwenhuijze et al., Ann. Rheum. Dis. (2003) 62:10-14), but are also used for the measurement of circulating cell free nucleosomes in serum and plasma (Holdenrieder et al. (2001)). Cell free serum and plasma nucleosome levels released into the circulation by dying cells have been measured by ELISA methods in studies of a number of different cancers to evaluate their use as a potential biomarker.

The cell free nucleosome may be mononucleosomes, oligonucleosomes, a constituent part of a larger chromatin fragment or a constituent part of a NET or a mixture thereof.

Mononucleosomes and oligonucleosomes can be detected by Enzyme-Linked ImmunoSorbant Assay (ELISA) and several methods have been reported (e.g. Salgame et al. (1997); Holdenrieder et al. (2001); van Nieuwenhuijze et al. (2003)). These assays typically employ an anti-histone antibody (for example anti-H2B, anti-H3 or anti-H1, H2A, H2B, H3 and H4) as capture antibody and an anti-DNA or anti-H2A-H2B-DNA complex antibody as detection antibody.

Circulating nucleosomes are not a homogeneous group of protein-nucleic acid complexes. Rather, they are a heterogeneous group of chromatin fragments originating from the digestion of chromatin on cell death and include an immense variety of epigenetic structures including particular histone isoforms (or variants), post-translational histone modifications, nucleotides or modified nucleotides, and protein adducts. It will be clear to those skilled in the art that an elevation in nucleosome levels will be associated with elevations in some circulating nucleosome subsets containing particular epigenetic signals including nucleosomes comprising particular histone isoforms (or variants), comprising particular post-translational histone modifications, comprising particular nucleotides or modified nucleotides and comprising particular protein adducts. Assays for these types of chromatin fragments are known in the art (for example, see WO 2005/019826, WO 2013/030579, WO 2013/030578, WO 2013/084002 which are herein incorporated by reference).

A number of proteins occur in NETs that are adducted directly or indirectly to nucleosomes. These proteins include, without limitation, myeloperoxidase (MPO), neutrophil elastase (NE), lactotransferrin, azurocidin, cathepsin G, leukocyte proteinase 3, lysozyme C, neutrophil defensin 1, neutrophil defensin 3, myeloid cell nuclear differentiation antigen, S100 calcium-binding protein A8, S100 calcium-binding protein A9, S100 calcium-binding protein A12, actin β, actin γ, alpha-actin, plastin-2, cytokeratin-10, catalase, alpha-enolase and transketolase (Urban et al., PLOS Pathogens. (2009) 10: e1000639). Any nucleosome-protein adduct that occurs in NETs is a useful adduct for the detection of elevated levels of NETs in methods of the invention. C-reactive protein (CRP) may also be adducted to nucleosomes in NETs and nucleosome-CRP adduct is therefore a useful adduct for the detection of elevated levels of NETs in methods of the invention.

In preferred embodiments of the invention the adduct used is a MPO-nucleosome adduct or a NE-nucleosome adduct.

In one embodiment, the component of the cell free nucleosome comprises an epigenetic feature of the cell free nucleosome.

The biomarker used in the methods of the invention may be the level of cell free nucleosomes per se and/or an epigenetic feature of a cell free nucleosome. It will be understood that the terms “epigenetic signal structure” and “epigenetic feature” are used interchangeably herein. They refer to particular features of the nucleosome that may be detected. In one embodiment, the epigenetic feature of the nucleosome is selected from the group consisting of: a post-translational histone modification, a histone isoform, a modified nucleotide and/or proteins bound to a nucleosome in a nucleosome-protein adduct.

In one embodiment, the epigenetic feature of the nucleosome comprises one or more histone variants or isoforms. The epigenetic feature of the cell free nucleosome may be a histone isoform, such as a histone isoform of a core nucleosome, in particular a histone H3 isoform. The term “histone variant” and “histone isoform” may be used interchangeably herein. The structure of the nucleosome can also vary by the inclusion of alternative histone isoforms or variants which are different gene or splice products and have different amino acid sequences. Many histone isoforms are known in the art. Histone variants can be classed into a number of families which are subdivided into individual types. The nucleotide sequences of a large number of histone variants are known and publicly available for example in the National Human Genome Research Institute NHGRI Histone Database (Mariño-Ramírez et al. The Histone Database: an integrated resource for histones and histone fold-containing proteins. Database Vol. 2011, and http://genome.nhgri.nih.gov/histones/complete.shtml), the GenBank (NIH genetic sequence) Database, the EMBL Nucleotide Sequence Database and the DNA Data Bank of Japan (DDBJ). For example, variants of histone H2 include H2A1, H2A2, mH2A1, mH2A2, H2AX and H2AZ. In another example, histone isoforms of H3 include H3.1, H3.2, H3.3 and H3t.

In one embodiment, the histone isoform is H3.1.

The structure of nucleosomes can vary by post translational modification (PTM) of histone proteins. PTM of histone proteins typically occurs on the tails of the core histones and common modifications include acetylation, methylation or ubiquitination of lysine residues as well as methylation or citrullination of arginine residues and phosphorylation of serine residues and many others. Many histone modifications are known in the art and the number is increasing as new modifications are identified (Zhao and Garcia (2015) Cold Spring Harb Perspect Biol, 7: a025064). Therefore, in one embodiment, the epigenetic feature of the cell free nucleosome may be a histone post translational modification (PTM). The histone PTM may be present on a core nucleosome histone (e.g. H2A, H2B, H3 or H4), or a linker histone (e.g. H1 or H5). The histone PTM may be a histone PTM of a core nucleosome, e.g. H3, H2A, H2B or H4, in particular H3, H2A or H2B. In particular, the histone PTM is a histone H3 PTM. Examples of such PTMs are described in WO 2005/019826 and WO 2017/068359.

For example, the post translational modification may include acetylation, methylation, which may be mono-, di- or tri-methylation, phosphorylation, ribosylation, citrullination, ubiquitination, hydroxylation, glycosylation, nitrosylation, glutamination and/or isomerisation (see Ausio (2001) Biochem Cell Bio 79:693). In one embodiment, the histone PTM is methylation of a lysine residue. In a further embodiment, the methylation is of a histone 3 lysine residue. In a yet further embodiment, the histone PTM is selected from H3K4Me, H3K4Me2, H3K9Me, H3K9Me3, H3K27Me3 or H3K36Me3. In one embodiment, the histone PTM is acetylation of a lysine residue. In a further embodiment, the acetylation is of a histone 3 lysine residue. In a yet further embodiment, the histone PTM is selected from H3K9Ac, H3K14Ac, H3K18Ac or H3K27Ac. In another embodiment, the histone PTM is H4PanAc. In one embodiment, the histone PTM is phosphorylation of a serine residue. In a further embodiment, the phosphorylation is of an isoform X of histone 2A (H2AX) serine residue or phosphorylation of a histone 3 serine residue. In a yet further embodiment, the histone PTM is selected from pH2AX or H3S10Ph. In one embodiment, the histone PTM is selected from citrullination or ribosylation. In a further embodiment, the histone PTM is citrullinated H3 (H3cit) or citrullinated H4 (H4cit). In a further embodiment, the histone PTM is citrullination of a histone 3 arginine residue. In a yet further embodiment, the histone PTM is H3R8Cit. In one embodiment, the histone PTM is selected from the group consisting of: H3K4Me, H3K4Me2, H3K9Me, H3K9Me3, H3K27Me3, H3K36Me3, H3K9Ac, H3K14Ac, H3K18Ac, H3K27Ac, H4PanAc, pH2AX, H3S10Ph and H3R8Cit.

In one embodiment, the histone PTM is selected from citrullination or ribosylation. In a further embodiment, the histone PTM is H3 citrulline (H3cit) or H4 citrulline (H4cit). In a yet further embodiment, the histone PTM is H3cit.

In one embodiment, the histone PTM is ribosylation, also referred to as ADP-ribosylation. Post-translational histone ADP-ribosylation of nucleosomes occupying promoters of inflammatory response markers in macrophages is stimulated by exposure to lipopolysaccharides leading to elevated transcription and may have antiviral properties. Therefore, altered levels of circulating ADP-ribosylated nucleosomes released from macrophages are expected to be useful in methods of the invention.

A group or class of related histone post translational modifications (rather than a single modification) may also be detected. A typical example, without limitation, would involve a 2-site immunoassay employing one antibody or other selective binder directed to bind to nucleosomes and one antibody or other selective binder directed to bind the group of histone modifications in question. Examples of such antibodies directed to bind to a group of histone modifications would include, for illustrative purposes without limitation, anti-pan-acetylation antibodies (e.g. a Pan-acetyl H4 antibody [H4panAc]), anti-citrullination antibodies or anti-ubiquitin antibodies.

In one embodiment, the epigenetic feature of the nucleosome comprises one or more DNA modifications. In addition to the epigenetic signalling mediated by nucleosome histone isoform and PTM composition, nucleosomes also differ in their nucleotide and modified nucleotide composition. Some nucleosomes may comprise more 5-methylcytosine residues (or 5-hydroxymethylcytosine residues or other nucleotides or modified nucleotides) than other nucleosomes. In one embodiment, the DNA modification is selected from 5-methylcytosine or 5-hydroxymethylcytosine.

In one embodiment, the epigenetic feature of the nucleosome comprises one or more protein-nucleosome adducts or complexes. A further type of circulating nucleosome subset is nucleosome protein adducts. It has been known for many years that chromatin comprises a large number of non-histone proteins bound to its constituent DNA and/or histones. These chromatin associated proteins are of a wide variety of types and have a variety of functions including transcription factors, transcription enhancement factors, transcription repression factors, histone modifying enzymes, DNA damage repair proteins and many more. These chromatin fragments including nucleosomes and other non-histone chromatin proteins or DNA and other non-histone chromatin proteins are described in the art.

Another way the structure of nucleosomes may vary is by mutation. Therefore, in one embodiment, the epigenetic feature is a mutated histone. In a further embodiment, the mutation is in histone 3 (H3). In a yet further embodiment, the mutation in H3 is when lysine 27 is replaced by a methionine (H3K27M).

In one embodiment, the protein adducted to the nucleosome (and which therefore may be used as a biomarker) is selected from: a transcription factor, a High Mobility Group Protein or chromatin modifying enzyme. References to “transcription factor” refer to proteins that bind to DNA and regulate gene expression by promoting (i.e. activators) or suppressing (i.e. repressors) transcription. Transcription factors contain one or more DNA-binding domains (DBDs), which attach to specific sequences of DNA adjacent to the genes that they regulate. All of the circulating nucleosomes and nucleosome moieties, types or subgroups described herein may be useful in the present invention.

It will be understood that more than one epigenetic feature of cell free nucleosomes may be detected in methods and uses of the invention. Multiple biomarkers may be used as a combined biomarker. Therefore, in one embodiment, the use comprises more than one epigenetic feature of cell free nucleosomes as a combined biomarker. The epigenetic features may be the same type (e.g. PTMs, histone isoforms, nucleotides or protein adducts) or different types (e.g. a PTM in combination with a histone isoform). For example, a post-translational histone modification and a histone variant may be detected (i.e. more than one type of epigenetic feature is detected). Alternatively, or additionally, more than one type of post-translational histone modification is detected, or more than one type of histone isoform is detected. In one aspect, the use comprises a post-translational histone modification and a histone isoform as a combined biomarker in a sample, for the diagnosis, detection, treatment selection, prognostication or monitoring of a condition. In one embodiment, the combined biomarker is H3.1 and H3cit. In an alternative embodiment, the combined biomarker is H3.1 and H4cit.

The term “biomarker” means a distinctive biological or biologically derived indicator of a process, event, or condition. Biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

Biomarkers are also useful as companion diagnostic products for the selection of patients suitable for treatment by a particular therapy.

The methods of the invention are directed to assigning the patient with a risk of an adverse outcome. Adverse outcomes include mortality and/or an acute event requiring immediate medical care, for example, hospitalisation (i.e. hospital treatment) and/or surgery. For some patients the condition is a serious one that can be fatal is left untreated, as it can lead to organ failure.

Assigning the patient with a risk of an adverse outcome may assign a near- or short-term risk or may assign a medium-term risk. A near- or short-term risk includes wherein the patient may develop an adverse outcome within 30 days, such as within 2 weeks or 14 days, within 1 week or 7 days or within 5 days or less of presentation of symptoms or of a positive diagnosis. Such near- or short-term risk may also include wherein the patient may develop an adverse outcome within 30 days, such as within 2 weeks or 14 days, within 1 week or 7 days or within 5 days or less of performing the methods described herein. An example of a short term risk includes the development of NETs related complications requiring hospital treatment. A medium-term risk includes wherein the patient may develop an adverse outcome more than 30 days after presentation of symptoms, a positive diagnosis and/or the performing of methods as described herein.

Therefore, in another aspect of the invention, there is provided a method of identifying a subject with a disorder of hemostasis or thrombosis requiring hospital treatment, comprising:
(i) contacting a body fluid sample obtained from the subject with a binding agent to detect or measure the level of cell free nucleosomes or a component thereof; and
(ii) using the level of cell free nucleosomes detected to determine if the subject should be admitted to hospital for treatment.

It will be understood that methods of the invention may also be used to identify patients who do not require hospital treatment, i.e. using the level of cell free nucleosomes detected to determine if the subject should not be admitted to hospital for treatment. This mode of the invention would help to identify patients who can be discharged early if they have already been admitted to hospital.

Methods and uses described herein may be tested in body fluid samples, in particular blood, serum or plasma samples. Preferably, plasma samples are used. Plasma samples may be collected in collection tubes containing one or more anticoagulants such as ethylenediamine tetraacetic acid (EDTA), heparin, or sodium citrate, in particular EDTA.

### VTE

The methods of the current invention find particular use in diagnosing and managing VTE, including DVT and PE.

Venous thromboembolism (VTE) refers to the abnormal coagulation of blood in the veins, resulting in complete or incomplete occlusion of blood vessels, which is a venous return disease, including deep vein thrombosis (DVT) and pulmonary thromboembolism (PTE), both of which are manifestations of the same disease in different parts, but also different stages of the same disease. It has become one of the important causes of unanticipated deaths in hospitals with high incidence, high morbidity and high mortality, but is generally considered to be one of the most preventable in-hospital deaths.

### Vasculitis

The methods of the current invention find particular use in diagnosing and managing vasculitis.

Vasculitis is the name of a group of conditions that cause inflammation of the blood vessels. For ease of reference, vasculitis may also be referred to herein as a ‘disorder’ or a ‘condition’. There are many types of vasculitis and the present invention is applicable to these types which are described below in more detail:

Eosinophilic Granulomatosis with Polyangiitis (Churg-Strauss Syndrome)

Eosinophilic granulomatosis with polyangiitis, also called Churg-Strauss syndrome, is a type of vasculitis that mainly affects adults around 38 to 54. It can cause:
asthma
cold-like symptoms caused by allergies (allergic rhinitis)
a high temperature
muscle and joint pain
tiredness
loss of appetite and weight loss.

It can also affect the nerves, causing weakness, pins and needles or numbness, and sometimes damages the kidneys or heart muscle.

### Temporal Arteritis (Giant Cell Arteritis)

Temporal arteritis, also known as giant cell arteritis, is a type of vasculitis where the arteries at the side of the head (the temples) become inflamed.

It mostly affects adults over the age of 50 and can cause:
aching and soreness around the temples
jaw muscle pain while eating
headaches
double vision or vision loss.

Some people with temporal arteritis also get polymyalgia rheumatica (muscle pain and stiffness in the shoulders, neck and hips).

It can lead to serious problems like stroke and blindness if not treated quickly.

Granulomatosis with Polyangiitis (Wegener's Granulomatosis)

Granulomatosis with polyangiitis, also called Wegener's granulomatosis, is a type of vasculitis that affects blood vessels in the nose, sinuses, ears, lungs and kidneys.

It mainly affects middle-aged or older people and can cause:
a high temperature
night sweats
inflammation of the sinuses (sinusitis)
nosebleeds and crusting of the nose
shortness of breath and coughing up blood
kidney problems.

Granulomatosis with polyangiitis is a serious condition that can be fatal if left untreated, as it can lead to organ failure.

### Henoch-Schonlein Purpura

Henoch-Schonlein purpura is a rare type of vasculitis that can affect the skin, kidneys or bowel. Children often get this type of vasculitis and it is thought to be triggered by the body reacting to an infection. It can cause:
a rash that looks like small bruises or reddish-purple spots (this may be less obvious on brown or black skin)
joint pain
tummy (abdominal) pain
diarrhoea
being sick
blood in urine or faeces.

### Kawasaki Condition

Kawasaki condition is a condition that mainly affects children under the age of 5.

The characteristic symptoms are a high temperature that lasts for 5 days or more, and possibly 1 or more of the following symptoms:
a rash
swollen glands in the neck
dry, cracked lips
red fingers or toes
red eyes.

### Microscopic Polyangiitis

Microscopic polyangiitis is a rare and potentially serious long-term type of vasculitis that most often develops in middle-aged people.

It can affect any organ, but often affects the lungs, kidneys and nerves. It can cause:
a rash
shortness of breath and coughing up blood
red and sore eyes
pins and needles or numbness
joint stiffness
muscle aches
loss of appetite
losing weight without trying
feeling tired
flu-like symptoms, such as a high temperature and an aching body
kidney problems.

### Polyarteritis Nodosa

Polyarteritis nodosa is a rare type of vasculitis that particularly affects the arteries supplying the gut, kidneys and nerves. It tends to develop in middle-aged people.

It can sometimes be triggered by an infection, such as hepatitis B, but the exact cause is uncertain. It can cause:
muscle and joint pain
tummy (abdominal) pain, particularly after eating
a rash
pins and needles or numbness
bleeding and ulcers in the gut.

Polyarteritis nodosa can be very serious if it is not treated.

### Polymyalgia Rheumatica

Polymyalgia rheumatica is a type of vasculitis that is closely related to temporal arteritis. It mostly affects adults over 50 and is more common in women than men.

It can cause:
pain and stiffness in the shoulders, neck and hips, which is often worse after waking up
a high temperature
extreme tiredness
loss of appetite and weight loss
depression.

### Takayasu Arteritis

Takayasu arteritis is a type of vasculitis that mainly affects young women. It affects the main artery from the heart, as well as the major arteries branching off it.

It can cause:
extreme tiredness
a high temperature
weight loss
muscle and joint pain
painful, numb or cold limbs.

Other types of vasculitis include:

### Behçet's Condition

Behçet's condition typically causes mouth ulcers and genital ulcers, and is more common in people from Greece, Turkey, the Middle East, China and Japan.

### Buerger's Condition

Buerger's condition affects blood vessels in the legs and arms, leading to reduced blood flow to the hands and feet. It is closely linked to smoking.

### Cogan's Syndrome

Cogan's syndrome is inflammation of the blood vessels in the inner ears and eyes.

### Cryoglobulin-Associated Vasculitis

Cryoglobulin-associated vasculitis is caused by abnormal proteins in the blood called cryoglobulins.

Cryoglobulin-associated vasculitis can sometimes happen after a hepatitis C infection and causes a rash on the lower limbs, joint pain, nerve damage, tummy (abdominal) pain and kidney problems.

### Hypersensitivity Vasculitis

Hypersensitivity vasculitis is usually caused by a reaction to a medicine, such as NSAIDs or certain antibiotics, and results in a temporary rash.

### Primary Angiitis of the Central Nervous System

Primary angiitis of the central nervous system is inflammation of the blood vessels in the brain.

### Rheumatoid Vasculitis

Rheumatoid vasculitis is vasculitis associated with rheumatoid arthritis.

In one embodiment, the vasculitis is selected from the group consisting of: eosinophilic granulomatosis with polyangiitis (Churg-Strauss syndrome), temporal arteritis (giant cell arteritis), granulomatosis with polyangiitis (Wegener's granulomatosis), Henoch-Schoenlein purpura, Kawasaki condition, microscopic polyangiitis, polyarteritis nodosa, polymyalgia rheumatica, Takayasu arteritis, Behcet's condition, Buerger's condition, Cogan's syndrome, Cryoglobulin-associated vasculitis, hypersensitivity vasculitis, primary angiitis of the central nervous system and rheumatoid vasculitis.

### DIC

The methods of the current invention find particular use in diagnosing and managing DIC. Disseminated intravascular coagulation (DIC) is an acquired syndrome characterised by activation of coagulation pathways, resulting in formation of intravascular thrombi and depletion of platelets and coagulation factors. Thrombi may lead to vascular obstruction/ischaemia and multi-organ failure. Spontaneous bleeding may occur. For ease of reference, DIC may also be referred to herein as a ‘disorder’ or a ‘condition’ in addition to a ‘syndrome’.

DIC is thus a consequence of dysregulated coagulation. DIC can be triggered by (i.e. is associated with or is secondary to) major trauma, organ destruction, sepsis or severe infection (including severe coronavirus disease 2019 [COVID-19] infection), severe obstetric disorders, some malignancies, major vascular disorders, and severe toxic or immunological reactions. The most common triggers are burns, sepsis, malignancy, and pregnancy.

In more detail, medical conditions that may be complicated by or associated with DIC include infections, especially when there is sepsis, which is the most common cause. Gram-negative bacteria are the most common triggers in this category. Other infections include Escherichia coli O157, typhoid fever, Rocky Mountain spotted fever and parasites.

Other medical conditions that may be complicated by or associated with DIC include malignancy, especially leukaemias; major trauma including crush syndrome and, occasionally, burns; complications of pregnancy including the placental problem of placental abruption, amniotic fluid embolism, severe hypertension of pregnancy with fulminating pre-eclampsia and HELLP syndrome; incompatible blood transfusion; transplant rejection; severe liver disease; pancreatitis; heat stroke; dissecting aortic aneurysm; complications after surgery; recreational drugs; snake bites; and connective tissue disorders, including antiphospholipid syndrome.

In one embodiment, the DIC is associated with trauma, organ destruction, sepsis or infection (including coronavirus disease 2019 [COVID-19] infection), obstetric disorders, malignancies, vascular disorders, and toxic or immunological reactions.

Unfortunately, no single laboratory test can be used to diagnose DIC. Instead, conventionally, a combination of a prolonged activated partial thromboplastin time (aPTT) and prothrombin time (PT), decreased fibrinogen, decreased platelets, increased D-dimer or fibrin degradation products, and schistocytes on a blood smear in an appropriate clinical context may suggest the diagnosis.

There may be issues associated with convention tests. For example, early in the course of DIC, the platelet count may be in the normal range. The finding of schistocytes is neither sensitive nor specific, and schistocytes are present in only 10% to 50% of cases of acute DIC. There are also conditions which make interpretation of laboratory abnormalities in DIC more difficult.

Fibrinogen is an acute-phase reactant. As such, it can be within the normal range in occasional patients with acute DIC, especially if an underlying inflammatory disorder exists; comparison with a baseline value would therefore be useful. In addition, the D-dimer can be chronically elevated in patients with cancer and in those with recent clots or liver disease.

It is also difficult to make the diagnosis of DIC in patients with advanced liver disease. The liver produces most coagulation proteins, and most cases of advanced liver disease are characterized by a prolonged PT and aPTT with decreased fibrinogen. The liver is also responsible for clearing D-dimers, which are therefore often elevated in liver disease. Additionally, the platelet count may be reduced in patients with cirrhosis due to hypersplenism and reduced production of thrombopoietin by the liver.

### APS

The methods of the current invention find particular use in diagnosing and managing antiphospholipid syndrome (APS). For ease of reference, APS may also be referred to herein as a ‘disorder’ or a ‘condition’ in addition to a ‘syndrome’.

Antiphospholipid syndrome (APS) is a prothrombotic condition characterized by venous or arterial thrombosis and/or pregnancy morbidity in the presence of persistent laboratory evidence of antiphospholipid antibodies (aPLs). aPLs are autoantibodies that target phospholipid-bound proteins, notably β₂-glycoprotein I (β₂GPI). Although the presence of these antibodies is the defining feature of this syndrome, the mechanism by which aPLs result in a hypercoagulable state remains incompletely understood. APS is recognized to be a syndrome prone to recurrent thrombosis when anticoagulants are discontinued, although some patients develop recurrent events despite standard anticoagulant therapy. Therefore, in one embodiment, the APS is associated with thrombosis.

Antiphospholipid antibodies involved in APS include anticardiolipin antibodies and lupus anticoagulants. Lupus anticoagulants have shown the closest association with thrombosis. Accordingly, methods of the invention may be used to detect the prothrombotic effect of lupus anticoagulants (LAs).

A subset of patients with APS have a severe variant known as catastrophic antiphospholipid syndrome (CAPS), which is characterized by thrombosis affecting multiple organs in a short period of time and histopathologic evidence of small vessel occlusion. Therefore, in one embodiment, the APS is CAPS.

Unfortunately to-date there has been no single test to detect lupus anticoagulant. Instead conventionally a combination of tests is needed. An example of a sequential nonstandard test which exists that helps determine whether a patient has lupus anticoagulants is:
1) Initial testing, which is also called the screening test, involves the use of one or more phospholipid-containing reagents. This test usually uses PTT, LA-sensitive PTT or dilute Russell viper venom test (DRVVT). These tests allow measuring the time it takes (in seconds) for a plasma sample to clot and which due to lupus anticoagulant gets prolonged beyond the upper limit of the reference.
2) Upon observation of prolongation in the above test, a mixing study is next. A mixing study is a mixture of an equal volume of patient's plasma with “normal” pooled plasma, and a PTT or preferably, DRVVT is performed on this mixture. Mixing study eliminates the possibility that prolongation is due to coagulation factor deficiency and confirms that an inhibitor is present. Mixing study also confirms that inhibitor is directed towards negatively charged phospholipid and not specific coagulation factor. The mixing study fails to correct the prolonged coagulation test if it is due to LA.
3) Additionally, a confirmatory test involves the addition of excess phospholipid to shorten or correct the prolonged coagulation test. Thus, lupus anticoagulant is characterized by the correction of prolonged clotting time with added phospholipid and not with control plasma. Many different sources of phospholipid are available, including aged platelets and synthetic phospholipids in the combination of APTT or DRVVT.

The International Society of Thrombosis and Hemostasis (ISTH) states that four criteria must be met to confirm the presence of lupus anticoagulant.
1. (Screening test) Prolonged result in one of two coagulation tests that are phospholipids dependent such as PTT-LA or DRVVT
2. (Mixing study) observe the prolonged result on mixing study
3. (Confirmatory test) Lack of prolonged time when adding additional phospholipids
4. Ruling out other coexisting coagulation factor inhibitor such as factor VII.

Lupus anticoagulant testing is often done simultaneously with cardiolipin antibody and anti-beta2-glycoprotein I antibodies when the antiphospholipid syndrome is suspected. Despite current guidelines and recommendations, many issues remain including the lack of uniformity among laboratories with testing protocols and procedures that overall impact result interpretations. Integrated test that consists of only screening and the confirmatory test has been used to detect LA with convenience and less time consumption. However, further investigation into the accuracy of these tests is still needed. In conclusion, LA diagnosis remains challenging and needs standardization.

The present invention aims to provide a test which can be used in diagnose and monitor APS, particularly the prothrombotic effects of LA, either as a single test or as part of a panel of tests. This panel of tests may include one of more of the tests mentioned above, or any other test which is available or becomes available for use in assisting in the diagnosis of APS.

### Measuring, Diagnosis and Monitoring Methods

The present invention aims to provide a test which can be used in diagnose and monitor a disorder of hemostasis or thrombosis, e.g. VTE, including DVT and/or PE, vasculitis, DIC or APS (particularly the prothrombotic effects of LA), either as a single test or as part of a panel of tests. Therefore, there is provided a method of diagnosing a disorder of hemostasis or thrombosis which comprises the step of detecting the level of cell free nucleosomes or a component thereof in a sample obtained from a subject.

In one embodiment, circulating nucleosome levels are measured in a sample taken from a subject suffering from a condition associated with a disorder of hemostasis or thrombosis to determine the prognosis of the condition. In another embodiment, circulating nucleosome levels are measured in multiple samples taken at intervals from a subject suffering from a condition associated with a disorder of hemostasis or thrombosis to monitor the progress of the condition and/or to assess the efficacy of treatment. In addition, within APS, repeat testing is needed over time to confirm the presence of antiphospholipid antibodies (aPL) because antibodies can appear with infections, but go away once the infection resolves.

In a further embodiment, circulating nucleosome levels are measured in a sample taken from a subject suffering from a disorder of hemostasis or thrombosis, in particular to assess the prognosis of the condition. Further measurements on multiple samples taken at intervals from a subject suffering from a disorder of hemostasis or thrombosis may be made to monitor the progress of the condition and/or to assess the efficacy of treatment.

Within DIC, laboratory parameters, including the PT, aPTT, fibrinogen, and platelet count, can be followed and should return to baseline levels as the disorder is effectively treated. In addition, clinical bleeding should improve. The D-dimer should also decline with treatment but may remain persistently elevated from other causes (unresolved clot, liver failure). The time course of recovery from DIC is usually linked to recovery from the underlying illness.

Identifying individuals at high risk of severe reaction or a complication, including organ failure, would allow triaging and facilitate allocation of strained medical resources. Therefore, in an embodiment there is provided a method of identifying a subject with a disorder of hemostasis or thrombosis requiring medical treatment, comprising:
(i) contacting a body fluid sample obtained from the subject with a binding agent to detect or measure the level of cell free nucleosomes or a component thereof; and
(ii) using the level of cell free nucleosomes detected to determine if the subject requires medical treatment.

According to a further aspect, there is provided a method of monitoring the severity of a disorder of hemostasis or thrombosis in a subject, comprising:
(i) contacting a body fluid sample obtained from the subject with a binding agent to detect or measure the level of cell free nucleosomes or a component thereof;
(ii) repeating the detection or measurement of the level of cell free nucleosomes or a component thereof in a body fluid obtained from the subject on one or more occasions;
(iii) using any changes in the level of cell free nucleosomes or component thereof to monitor the progression of the disorder in the subject.

According to a further aspect, there is provided a method for monitoring the progression of the condition in a subject having or suspected of having a disorder of hemostasis or thrombosis, or being predisposed to a poor prognosis, which comprises the steps of:
(i) contacting a sample obtained from the subject with a binding agent to detect or measure the level of cell free nucleosomes; and
(ii) comparing the level of cell free nucleosomes detected with an earlier sample taken from said subject to monitor the progression of the condition.

According to a further aspect, there is provided a method of monitoring the progress of a condition in a subject suffering from a disorder of hemostasis or thrombosis, comprising:
(i) contacting a body fluid sample obtained from the subject with a binding agent to detect or measure the level of cell free nucleosomes or a component thereof;
(ii) repeating step (i) on one or more occasions; and
(iii) using any changes in the level of cell free nucleosomes or component thereof to monitor the progression of the condition in the subject.

If a subject is determined to not have a disorder of hemostasis or thrombosis or to have a mild complication, then the invention may still be used for the purposes of monitoring condition progression for future development of a medical complication. For example, the biomarker level measurements can be repeated at another time point to establish if the biomarker level has changed.

Detecting and/or quantifying may be performed directly on the purified or enriched nucleosome sample, or indirectly on an extract therefrom, or on a dilution thereof. Quantifying the amount of the biomarker present in a sample may include determining the concentration of the biomarker present in the sample. Uses and methods of detecting, monitoring and of diagnosis according to the invention described herein are useful to confirm the existence of a condition, to monitor development of the condition by assessing onset and progression, or to assess amelioration or regression of the condition. Uses and methods of detecting, monitoring and of diagnosis are also useful in methods for assessment of clinical screening, prognosis, choice of therapy, evaluation of therapeutic benefit, i.e. for drug screening and drug development.

In one embodiment the condition is a condition involving pathological clinical complications of high levels of NETs or NETosis.

The detection or measurement may comprise an immunoassay, immunochemical, mass spectroscopy, chromatographic, chromatin immunoprecipitation or biosensor method. In particular, detection and/or measurement may comprise a 2-site immunoassay method for nucleosome moieties. Such a method is preferred for the measurement of nucleosomes or nucleosome incorporated epigenetic features in situ employing two anti-nucleosome binding agents or an anti-nucleosome binding agent in combination with an anti-histone modification or anti-histone variant or anti-DNA modification or anti-adducted protein detection binding agent. Also, detection and/or measurement may comprise a 2-site immunoassay, for example employing combinations of a labelled or immobilized: anti-nucleosome, anti-histone modification, anti-histone variant/isoform, anti-DNA modification or anti-adducted protein binding agent.

The inventors herein used a 2-site immunoassay for H3.1-nucleosomes employing an immobilized anti-histone H3.1 antibody directed to bind to an epitope around amino acids 30-33 of the histone H3.1 protein to capture clipped and non-clipped nucleosomes, together with a labelled anti-nucleosome antibody directed to bind to an epitope present in intact nucleosomes but not present on isolated (free) histone or DNA nucleosome components. This type of epitope may be referred to as a “conformational nucleosome epitope” herein because it requires the native three-dimensional configuration of the target nucleosome to be intact.

H3R8Cit nucleosome measurements described herein were performed using a 2-site immunoassay employing an immobilized antibody directed to bind to nucleosomes citrullinated at arginine 8 of histone H3, together with the same labelled anti-nucleosome antibody directed to bind to a conformational nucleosome epitope.

In one embodiment, the method of detection or measurement comprises contacting the body fluid sample with a solid phase comprising a binding agent that detects cell free nucleosomes or a component thereof, and detecting binding to said binding agent.

In one embodiment, the method of detection or measurement comprises: (i) contacting the sample with a first binding agent which binds to an epigenetic feature of a cell free nucleosome; (ii) contacting the sample bound by the first binding agent in step (i) with a second binding agent which binds to cell free nucleosomes; and (iii) detecting or quantifying the binding of the second binding agent in the sample.

In another embodiment, the method of detection or measurement comprises: (i) contacting the sample with a first binding agent which binds to cell free nucleosomes; (ii) contacting the sample bound by the first binding agent in step (i) with a second binding agent which binds to an epigenetic feature of the cell free nucleosome; and (iii) detecting or quantifying the binding of the second binding agent in the sample.

Detecting or measuring the level of the biomarker(s) may be performed using one or more reagents, such as a suitable binding agent. For example, the one or more binding agents may comprise a ligand or binder specific for the desired biomarker, e.g. nucleosomes or component part thereof, an epigenetic feature of a nucleosome, a structural/shape mimic of the nucleosome or component part thereof, optionally in combination with one or more interleukins.

It will be clear to those skilled in the art that the terms “antibody”, “binder” or “ligand” as used herein are not limiting but are intended to include any binder capable of binding to particular molecules or entities and that any suitable binder can be used in the method of the invention. It will also be clear that the term “nucleosomes” is intended to include mononucleosomes, oligonucleosomes, NETs and any protein-DNA chromatin fragments that can be analysed in fluid media. In one embodiment, the binding agent, such as the antibody, specifically binds to the target biomarker. The specificity of an antibody is the ability of the antibody to recognize a particular antigen as a unique molecular entity and distinguish it from another. An antibody that “specifically binds” to an antigen or an epitope is a term well understood in the art. A molecule is said to exhibit “specific binding” if it reacts more frequently, more rapidly, with greater duration and/or with greater affinity with a particular target antigen or epitope, than it does with alternative targets. An antibody “specifically binds” to a target antigen or epitope if it binds with greater affinity, avidity, more readily, and/or with greater duration than it binds to other substances.

Methods of detecting biomarkers are known in the art. The reagents may comprise one or more ligands or binders, for example, naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

The term “detecting” or “diagnosing” as used herein encompasses identification, confirmation, and/or characterisation of a condition state. Methods of detecting, monitoring and of diagnosis according to the invention are useful to confirm the existence of a condition, to monitor development of the condition by assessing onset and progression, or to assess amelioration or regression of the condition. Methods of detecting, monitoring and of diagnosis are also useful in methods for assessment of clinical screening, prognosis, choice of therapy, evaluation of therapeutic benefit, i.e. for drug screening and drug development.

Methods of the invention may involve normalisation of marker levels. For example, the level of cell free nucleosomes containing a particular epigenetic feature may be normalised against the level of nucleosomes per se (or some other type of nucleosomes or parameter) to express the level as a proportion of nucleosomes containing the feature. For example, to express the level of citrullinated nucleosomes as the proportion of nucleosomes that are citrullinated.

In one embodiment, the method described herein is repeated on multiple occasions. This embodiment provides the advantage of allowing the detection results to be monitored over a time period. Such an arrangement will provide the benefit of monitoring or assessing the efficacy of treatment of a condition state. Such monitoring methods of the invention can be used to monitor onset, progression, stabilisation, amelioration, relapse and/or remission. In the context of APS, patients may be monitored over a period of 12 weeks or more, i.e. the method may be repeated after at least 12 weeks to confirm the persistence of the disorder.

In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the nature or amount of the biomarker(s) in test samples taken on different occasions.

A change in the level of the biomarker in the test sample relative to the level in a previous test sample taken earlier from the same test subject may be indicative of a beneficial effect, e.g. stabilisation or improvement, of said therapy on the disorder or suspected disorder. Furthermore, once treatment has been completed, the method of the invention may be periodically repeated in order to monitor for the recurrence of a disorder or condition.

Methods for monitoring efficacy of a therapy can be used to monitor the therapeutic effectiveness of existing therapies, such as steroid treatment, and new therapies in human subjects and in non-human animals (e.g. in animal models). These monitoring methods can be incorporated into screens for new drug substances and combinations of substances.

In a further embodiment the monitoring of more rapid changes due to fast acting therapies may be conducted at shorter intervals of hours or days.

Diagnostic or monitoring kits (or panels) are provided for performing methods of the invention. Such kits will suitably comprise one or more ligands for detection and/or quantification of the biomarker according to the invention, and/or a biosensor, and/or an array as described herein, optionally together with instructions for use of the kit.

Therefore according to a further aspect of the invention, there is provided a kit comprising reagents for use in the method as defined herein. According to a further aspect, there is provide the use of a kit comprising one or more reagents to detect or measure the level of cell free nucleosomes or a component thereof, to detect, monitor or diagnose a disorder of hemostasis or thrombosis

A further aspect of the invention is a kit for detecting the presence of a disorder of hemostasis or thrombosis comprising a biosensor capable of detecting and/or quantifying one or more of the biomarkers as defined herein. As used herein, the term “biosensor” means anything capable of detecting the presence of the biomarker. Examples of biosensors are described herein. Biosensors may comprise a ligand binder or ligands, as described herein, capable of specific binding to the biomarker. Such biosensors are useful in detecting and/or quantifying a biomarker of the invention.

According to a further aspect of the invention, there is provided a kit for assessing the health of a subject, wherein said kit comprises a first binding agent which specifically binds to an epigenetic feature of a cell free nucleosome (e.g. H3.1) and a second binding agent which specifically binds to cell free nucleosomes.

Suitably, biosensors for detection of one or more biomarkers combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace. Biosensors to detect one or more biomarkers of the invention include acoustic, plasmon resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more biomarkers.

Biomarkers for detecting the presence of a condition are essential targets for discovery of novel targets and drug molecules that retard or halt progression of the condition. As the level of the biomarker is indicative of condition and of drug response, the biomarker is useful for identification of novel therapeutic compounds in in vitro and/or in vivo assays. Biomarkers described herein can be employed in methods for screening for compounds that modulate the activity of the biomarker.

Thus, in a further aspect of the invention, there is provided the use of a binder or ligand, as described, which can be a peptide, antibody or fragment thereof or aptamer or oligonucleotide directed to a biomarker according to the invention; or the use of a biosensor, or an array, or a kit according to the invention, to identify a substance capable of promoting and/or of suppressing the generation of the biomarker.

The term “biomarker” means a distinctive biological or biologically derived indicator of a process, event, or condition. Biomarkers can be used in methods of diagnosis, e.g. clinical screening, and prognosis assessment and in monitoring the results of therapy, identifying subjects most likely to respond to a particular therapeutic treatment, drug screening and development. Biomarkers and uses thereof are valuable for identification of new drug treatments and for discovery of new targets for drug treatment.

The immunoassays described herein include any method employing one or more antibodies or other specific binders directed to bind to the biomarkers defined herein. Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels. All of said immunoassay methods are well known in the art, see for example Salgame et al. (1997) and van Nieuwenhuijze et al. (2003).

Identifying, detecting and/or quantifying can be performed by any method suitable to identify the presence and/or amount of a specific protein in a biological sample from a subject or a purification or extract of a biological sample or a dilution thereof. In particular, quantifying may be performed by measuring the concentration of the target in the sample or samples. Biological samples that may be tested in a method of the invention include those as defined hereinbefore. The samples can be prepared, for example where appropriate diluted or concentrated, and stored in the usual manner. The present invention finds particular use in plasma samples which may be obtained from the subject.

The term “detecting” or “diagnosing” as used herein encompasses identification, confirmation, and/or characterisation of a disease state, degeneration state or health state of an individual or an organ. Methods of detecting, monitoring and of diagnosis according to the invention are useful to confirm the health or condition of a subject or an organ, to monitor development of the condition by assessing onset and progression, or to assess amelioration or regression of the condition. Methods of detecting, monitoring and of diagnosis are also useful in methods for assessment of clinical screening, prognosis, choice of therapy, evaluation of therapeutic benefit, i.e. for drug screening and drug development.

Identification, detection and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

For example, detecting and/or quantifying can be performed by one or more method(s) selected from the group consisting of: immunoassay, immunochromatography, SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving detection and/or quantification of one or more biomarkers of the invention can be performed on bench-top instruments, or can be incorporated onto disposable, diagnostic or monitoring platforms that can be used in a non-laboratory environment, e.g. in the physician's office or at the subject's bedside. Suitable biosensors for performing methods of the invention include “credit” cards with optical or acoustic readers. Biosensors can be configured to allow the data collected to be electronically transmitted to the physician for interpretation and thus can form the basis for e-medicine. Therefore, in a further aspect of the invention, there is provided the use of a near patient or point-of-care immunoassay method for the measurement of a biomarker according to the invention. In one embodiment the near patient immunoassay method comprises a point-of-care immunoassay instrument (e.g. the Abbott i-STAT or the LightDeck Diagnostics point-of-care immunoassay instrument). In one embodiment the near patient immunoassay method comprises a lateral flow test. In a preferred embodiment the biomarker is a nucleosome or a nucleosome containing an epigenetic feature.

The identification of biomarkers for a condition state permits integration of diagnostic procedures and therapeutic regimes. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus, by monitoring a biomarker of the invention, subject care can be tailored precisely to match the needs determined by the disorder and the pharmacological profile of the subject, the biomarker can thus be used to titrate the optimal dose, predict a positive therapeutic response and identify those subjects at high risk of severe side effects.

Biomarker-based tests provide a first line assessment of ‘new’ subjects, and provide objective measures for accurate and rapid diagnosis, not achievable using the current measures.

Biomarker monitoring methods, biosensors, point-of-care tests, lateral flow tests and kits are also vital as subject monitoring tools, to enable the physician to determine whether relapse is due to worsening of the condition. If pharmacological treatment is assessed to be inadequate, then therapy can be reinstated or increased; a change in therapy can be given if appropriate. As the biomarkers are sensitive to the state of the condition, they provide an indication of the impact of drug therapy.

References to “subject”, “individual” or “patient” are used interchangeably herein. The subject may be a human or an animal subject. In one embodiment, the subject is a human. In one embodiment, the subject is a (non-human) animal. In one embodiment, the subject is suffering from a condition associated with high levels of lupus anticoagulant, such as autoimmune diseases (for example, systemic lupus erythematosus) or infections (for example Epstein-Barr virus, syphilis and hepatitis C). In one embodiment, the subject is a pregnant female.

In some embodiments the invention encompasses animal subjects (wild or domesticated). In some embodiments, the invention relates to veterinary uses including for livestock and companion animals such as cats, dogs, horses, sheep, goats, pigs, deer, llamas, cows and cattle.

The panels and methods described herein may be performed in vitro, or ex vivo. The methods described herein are preferably performed in vitro. References to acts carried out on a body fluid sample “obtained” from a subject are intended to encompass acts carried only a body fluid sample already obtained of “obtainable” from a subject and vice versa.

Detecting and/or quantifying may be compared to a cut-off level. Cut-off values can be predetermined by analysing results from multiple patients and controls, and determining a suitable value for classifying a subject as with or without the condition. For example, for conditions where the level of biomarker is higher in patients suffering from the condition, then if the level detected is higher than the cut-off, the patient is indicated to suffer from the condition. Alternatively, for conditions where the level of biomarker is lower in patients suffering from the condition, then if the level detected is lower than the cut-off, the patient is indicated to suffer from the condition. The advantages of using simple cut-off values include the ease with which clinicians are able to understand the test and the elimination of any need for software or other aids in the interpretation of the test results. Cut-off levels can be determined using methods in the art.

Detecting and/or quantifying may also be compared to a control. It will be clear to those skilled in the art that the control subjects may be selected on a variety of basis which may include, for example, subjects known to be free of the condition or may be subjects with a different condition (for example, for the investigation of differential diagnosis). The “control” may comprise a healthy subject, and/or a subject without the condition. The control may also be a subject with the condition displaying no, or mild, symptoms. Mild symptoms may include manageable symptoms which do not require hospital intervention and/or intensive medical treatment.

In one embodiment, a subject who tests positive by methods of the invention may be afflicted with the condition and additionally suffers, or goes on to suffer, further medical complications. In contrast, a control subject may also be afflicted with a condition but does not suffer, and does not go on to suffer, medical complications. Comparison with a control is well known in the field of diagnostics. The range of values found in the control group may be used as a normal or healthy or reference range against which the values found for test subjects can be compared. For example, if the reference range is <10 units, then a test value of 5 units would be considered normal, or not in need of treatment, but a value of 11 units would be considered abnormal and indicative of a need for treatment.

Therefore, in one embodiment, the method additionally comprises comparing the level of cell free nucleosomes or component thereof in the body fluid sample of the subject with one or more controls. For example, the method may comprise comparing the level of cell free nucleosomes present in a sample obtained from the subject with the level of cell free nucleosomes present in a sample obtained from a normal subject. The control may be a healthy subject.

In one embodiment, the level of cell free nucleosomes or component thereof is elevated compared to the control.

It will be understood that it is not necessary to measure control levels for comparative purposes on every occasion. For example, for healthy controls, once the ‘normal range’ is established it can be used as a benchmark for all subsequent tests. A normal range can be established by obtaining samples from multiple control subjects without the condition and testing for the level of biomarker. Results (i.e. biomarker levels) for subjects suspected to have the condition can then be examined to see if they fall within, or outside of, the respective normal range. Use of a ‘normal range’ is standard practice for the detection of condition.

In one embodiment, the method additionally comprises determining at least one clinical parameter for the patient. This parameter can be used in the interpretation of results. Clinical parameters may include any relevant clinical information for example, without limitation, body temperature, gender, weight, Body Mass Index (BMI), smoking status and dietary habits. Therefore, in one embodiment, the clinical parameter is selected from the group consisting of: body temperature, age, sex and body mass index (BMI). In the context of VTE, additional parameters include clinical signs and symptoms of VTE, such as leg swelling and pain with palpation of the deep veins, heart rate of e.g. more than 100 beats per minute, assessment of level of recent immobilisation, previous VTE, haemoptysis, or malignancy.

In one embodiment, the method of the invention is performed to identify a subject at high risk of developing a severe reaction to the condition and therefore in need of medical intervention. Such medical intervention may include one or more of the therapies as described herein.

According to another aspect of the invention, there is provided the use of a binding agent in the manufacture of a kit for use in a method of assigning a risk of an adverse outcome to a subject suffering from a disorder of hemostasis or thrombosis comprising:
(i) contacting a body fluid sample obtained from the subject with the binding agent to detect or measure the level of cell free nucleosomes or a component thereof; and
(ii) using the level of cell free nucleosomes detected to assign the likelihood of an adverse outcome to said subject.

According to a further aspect of the invention, there is provided the use of a binding agent in the manufacture of a kit for use in a method of detecting a subject in need of medical treatment for a disorder of hemostasis or thrombosis comprising:
(i) contacting a body fluid sample obtained from the subject with the binding agent to detect or measure the level of cell free nucleosomes or a component thereof; and
(ii) using the level of cell free nucleosomes as an indicator that the subject is in need of medical treatment for a disorder of hemostasis or thrombosis

### Additional Biomarkers

The level of cell free nucleosomes may be detected or measured as one of a panel of measurements. In the context of DIC and APS, these tests or measurements may include one or more of the following: a complete blood count (CBC); partial thromboplastin time (PTT); prothrombin time (PT) test; fibrinogen blood test; fibrin degradation products (FDP) test; or D-dimer test. Other measurements may include soluble fibrin (SF) and levels of natural anticoagulants such as antithrombin (AT) and protein C (PC). Furthermore, in APS only these tests or measurements may include one of more of the standard tests for APS such as: a test to measure activated partial thromboplastin time (aPTT), LA sensitive aPTT (LA-aPTT) test, diluted or modified Russell viper venom screen (dRVVT, MRVVT), prothrombin time (PT) test, an assay for anticardiolipin antibody, and an assay for anti-beta2-glycoprotein antibody.

The present invention may also be used in combination with the ISTH DIC or the ISTH APS scoring system.

The panel may also comprise different epigenetic features of the nucleosome as described hereinbefore (e.g. a histone isoform and a PTM). Biomarkers useful in a panel test for the detection of a disorder of hemostasis or thrombosis that require medical intervention include, without limitation, cytokine moieties (particularly interleukins), C-reactive protein, myeloperoxidase, D-Dimer, factor VII-activating protease (FSAP), fibrinogen and fibrin/fibrinogen breakdown products. In one embodiment, the panel comprises C-reactive protein. In one embodiment, the panel comprises one or more cytokines, such as one or more interleukins.

Interleukins (ILs) are a group of cytokines, usually secreted by leukocytes, that act as signal molecules. They have key roles in stimulating immune responses and inflammation. They were first identified in the 1970s and have been designated numerically as more interleukin types have been discovered. Examples of interleukins include, but are not limited to: IL-1, IL-2, IL-3, IL-4, IL-5, IL-6, IL-7, IL-8, IL-9, IL-10, IL-11, IL-12, IL-13, IL-14 and IL-15.

In one embodiment, the one or more interleukins is selected from the group consisting of: Interleukin-6 (IL-6) and Interleukin-12 (IL-12).

The interleukin may be IL-6. Interleukin-6 (IL-6) is a cytokine with a wide variety of biological functions. It is a potent inducer of fever and the acute phase response. The sequence of human IL-6 is known in the art and is described at UniProt Accession No. P05231. In one particular embodiment, the interleukin may be IL-6.

Alternatively, or additionally, the interleukin may be IL-12. Interleukin-12 (IL-12) is a T cell stimulating factor because it stimulates the growth and function of T cells. It is a heterodimeric cytokine comprised of IL-12A and IL-12B. The sequence of human IL-12A is known in the art and is described at UniProt Accession No. P29459 and the sequence of human IL-12B is also known and described at UniProt Accession No. P29460. In one particular embodiment, the interleukin may be IL-12.

In one embodiment, the panel comprises a cell free nucleosome or an epigenetic feature thereof and an interleukin. In another embodiment, the panel comprises an epigenetic feature of a cell free nucleosome and two interleukins. For example, the cell free nucleosome measurement can be combined with more than one interleukin measurement, such as IL-6 and IL-12. In a further embodiment, the epigenetic feature of a cell free nucleosome is selected from a histone isoform, such as H3.1, and a post translationally modified histone, such as H3cit. In a yet further embodiment, the panel of measurements is H3.1, H3cit, H4cit and IL-6.

In one embodiment, the panel comprises C-reactive protein (CRP). CRP is a pentameric protein found in plasma and levels of CRP (whether or not adducted to nucleosomes) increase in plasma in response to inflammation, such as in bacterial, viral, fungal and microbial infections. CRP levels increase following IL-6 secretion by macrophages and T cells and its physiological role is to bind lysophosphatidylcholine expressed on the surface of dead or dying cells in order to activate the complement system via C1q. It also binds to phosphocholine on the surface of some bacteria and enhances phagocytosis. The measurement of CRP levels is useful for determining the progression of a condition and the effectiveness of treatments and elevated CRP levels have been shown in patients with increased risk of diabetes, hypertension and cardiovascular condition. Increased CRP levels have also been found in patients with kidney failure and inflammatory bowel condition (IBD, including Crohn's condition and ulcerative colitis) and roughly correlate with coronary heart condition, although as elevated CRP is not directly related to heart condition it is not a specific prognostic marker. Since CRP is increased during inflammation, a disorder of hemostasis or thrombosis may also lead to increased CRP levels in plasma.

In one embodiment (particularly in the context of VTE), the panel comprises P-selectin and/or thrombin generation.

In one embodiment, the panel comprises myeloperoxidase (MPO). MPO is expressed in neutrophil granulocytes and produces hypohalous acids to carry out their antimicrobial activity. It is stored in azurophilic granules and released into the extracellular space during degranulation. The levels of MPO have been shown to be a useful predictor for myocardial infarction and have been combined with measurement of CRP for increased accuracy in predicting myocardial infarction risk in patients. In one embodiment, the panel comprises neutrophil elastase (NE).

In a preferred embodiment the panel comprises a D-dimer test. The D-dimer test measures the final dimeric form of fibrin clot formation using an antigen-antibody reaction.

In the context of VTE, a preferred embodiment of the invention is where the diagnostic method is carried out in association with a Wells score. The Wells score is a known prediction score for estimating the probability of DVT or PE. There are a number of versions of Wells scores available. In one embodiment the 2-level DVT Wells score or the 2-Level PE Wells score is used.

In the context of VTE, one embodiment of the invention is where the diagnostic method is carried out in association with the PE rule-out criteria (PERC). PERC is intended to rule out patients who are considered low-risk for PE.

Models can be derived using the biomarkers of the invention. Methods for deriving models or algorithms are well known in the art and suitable software packages are available. Typical software tools for this purpose include SPSS (Statistical Package for the Social Sciences) and “R”. These software packages provide for linear and non-linear data modelling of clinical data.

It will be clear to those skilled in the art, that any combination of the tests and biomarkers disclosed herein may be used in panels and algorithms for the detection or prediction of a complication to the condition, and that further markers may be added to a panel including these markers.

According to an aspect of the invention there is provided the use of a panel test to detect or predict a complication to the disorder of hemostasis or thrombosis in a patient, wherein the panel test comprises reagents to detect measurements of nucleosomes or a component thereof and one or more interleukins, in a sample obtained from the patient. In one embodiment the complication is organ failure. In one embodiment the complication is a NETs associated complication. In one embodiment the complication is an embolism or thrombotic complication. In one embodiment the complication is ARDS, ARS, SARS or an embolism or thrombotic complication. In one embodiment the complication is trauma, organ destruction, sepsis or infection (including coronavirus disease 2019 [COVID-19] infection), obstetric disorders, malignancies, vascular disorders, and toxic or immunological reactions.

According to one aspect of the present invention there is provided use of an imaging technique to detect, assist in or confirm the diagnosis of VTE, usually after the method of the invention has been conducted. Such imaging techniques include computed tomography angiography (CTA), computed tomography angiography with venous imaging (CTAV), ventilation/perfusion (V/Q) scan, and ultrasound scan (US).

### Methods of Treatment

According to a further aspect, there is provided a method of treating a disorder of hemostasis or thrombosis in a subject, which comprises the following steps:
(i) detecting or measuring the level of cell free nucleosomes in a sample obtained from the subject;
(ii) using the level measured in step (i) as indicative of the presence and/or severity and/or a medical complication of said condition in the subject; and
(iii) administering a therapy if the subject is determined to have a severe version of the condition or a medical complication in step (ii).

According to a further aspect, there is provided a method of treating a disorder of hemostasis or thrombosis a subject in need thereof, which comprises the step of administering a therapy (e.g. a therapeutic agent) to a subject identified as having differing levels of cell free nucleosomes in a sample obtained from said subject, when compared to the level of cell free nucleosomes in a sample obtained from a control subject. The therapy may include one or more suitable treatments for the condition including e.g. anticoagulation drugs or anti-inflammatory drugs. The management of DIC will focus primarily on treatment of the underlying disorder. Supportive treatments ay include plasma transfusions, transfusions of red blood cells and/or platelets, and anti-coagulant medication. APS cannot yet be cured so instead the current treatment is aimed at preventing clotting. There are two types of drugs used for this: antiplatelet drugs (such as aspirin and clopidogrel) and anticoagulant drugs (such as heparin, warfarin and rivaroxaban). In very rare cases, blood clots can suddenly form throughout the body, resulting in multiple organ failure. This is known as catastrophic antiphospholipid syndrome (CAPS). CAPS requires immediate emergency treatment in hospital with high-dose anticoagulants.

In one embodiment, the treatment is an anti-coagulant drug such as heparins, such as low weight heparins (LMWHs) and unfractionated heparin (UFH), warfarin, dabigatran, rivaroxaban, apixaban and edoxaban.

Treatments also include surgical removal and inferior vena cava filters. A patient may also be prescribed surgical stockings and exercise.

Sometimes, VTE causes an aneurysm—a bulge or ballooning in the wall of a blood vessel. This bulge may need surgery to reduce the risk of it rupturing. Blocked arteries also may require surgical treatment to restore blood flow to the affected area.

In one embodiment, the treatment is an anti-inflammatory drug. Many steroidal and non-steroidal anti-inflammatory drugs are known in the art. Some examples of steroidal anti-inflammatory drugs include without limitation, dexamethasone, methotrexate, azathioprine, mycophenolate, cyclophosphamide, tocilizumab, rituximab, hydrocortisone, cortisone, betamethasone, prednisone, prednisolone, triamcinolone and methylprednisolone. Some examples of non-steroidal anti-inflammatory drugs include without limitation, aspirin, celecoxib, diclofenac, diflunisal, etodolac, ibuprofen, indomethacin, CD24Fc (CD24 protein attached to the Fc region of immunoglobulin G) and EXO-CD24 (CD24-Exosomes).

Sometimes, vasculitis causes an aneurysm—a bulge or ballooning in the wall of a blood vessel. This bulge may need surgery to reduce the risk of it rupturing. Blocked arteries also may require surgical treatment to restore blood flow to the affected area. Similarly radical treatment also be needed to deal with associated organ failure.

The methods may comprise:
(i) measuring the level of cell free nucleosomes (optionally in combination with the level of one or more aforementioned biomarkers) in a sample obtained from the subject;
(ii) identifying the subject as suffering from a disorder of hemostasis or thrombosis condition (such those indicated above) in need of treatment based on a higher level of cell free nucleosomes compared to a control; and
(iii) administering a treatment to the subject.

In one embodiment, there is a provided a method to identify a subject suffering from a disorder of hemostasis or thrombosis, including DVT or PE, who has, or is at risk of developing, a medical complication that requires treatment comprising the steps of:
(i) measuring the level of cell free nucleosomes (optionally in combination with the level of one or more biomarkers) in a sample obtained from the subject;
(ii) identifying the subject as suffering from the condition in need of treatment based on a higher level of cell free nucleosomes compared to a control; and
(iii) administering a treatment to the subject.

According to another aspect of the invention there is provided a method of treatment a disorder of hemostasis or thrombosis comprising identifying a patient in need of treatment for said condition using a panel test and providing said treatment, wherein the panel test comprises reagents to detect measurements of nucleosomes or components thereof. A patient with a disorder of hemostasis or thrombosis is expected to have a higher level of cell free nucleosomes compared to a control.

### Antibodies

The immunoassays described herein for the measurement of H3.1-nucleosomes, citrullinated nucleosomes, MPO and NE use high avidity and specificity monoclonal antibodies for binding to nucleosomes and NETs. These antibodies bind strongly and specifically to NETs, NETs metabolites and nucleosomes. These antibodies may therefore be used as therapeutic antibodies to bind to NETs in vivo to neutralise NETs and facilitate their clearance from the body, for example by phagocytosis (Weiskopf and Weissman, Mabs (2015) 7:303-10).

The anti-histone H3.1 antibody, anti-nucleosome antibody and anti-citrullinated H3 antibody used by the inventors for the assay of nucleosomes described herein, have been selected as highly avid and specific antibodies and are therefore particularly useful as therapeutic antibodies.

In particular, the anti-histone H3.1 antibody may be highly specific. Nucleosomes are subject to clipping in which the histone tail is physically and irreversibly removed by regulated proteolysis, or clipping. Furthermore, histone degradation has been shown to be involved in the formation of NETs (see Papayannopoulos et al. (2010) J. Cell Biol. 191 (3): 677-691). On histone H3, clipping is reported to occur around amino acid position 21 (Yi and Kim (2018) BMB Reports, 51 (5): 211-218). The amino acid sequence of histone H3.1 at positions 27-36 is KSAPATGGVK (SEQ ID NO: 1). The amino acid sequence at positions 29-35 does not include any commonly post-translationally modified amino acids (for example lysine, serine or arginine). Therefore, antibodies directed to bind to this epitope (i.e. amino acid positions 29-35) are unaffected, or minimally affected, by the post-translational modification status of the nucleosome, and will bind to all or most nucleosomes containing histone H3.1, regardless of PTM structure. Therefore, the solid phase capture antibody selected for use by the inventors for the immunoassay described herein, was an anti-histone H3.1 antibody directed to bind to an epitope located within the core of the histone near to amino acid position 30-33 so that both intact and clipped nucleosomes are captured by the antibody regardless of their PTM status. This maximises the capture of H3.1-nucleosomes. The amino acid sequence of histone H3.1 is known in the art and is described at UniProt Accession No. P68431.

Therefore, in one embodiment of the invention, the antibody is directed to bind to a core histone epitope of histone H3.1 at an amino acid epitope located higher than amino acid position 21. In a preferred embodiment, the anti-histone H3.1 antibody is directed to bind to an epitope located within the core of the histone H3, at or near to amino acid position 29-35, in particular at or near to amino acid position 30-33.

The labelled antibody used by the inventors herein for immunoassay can be an anti-nucleosome antibody directed to bind to a conformational nucleosome epitope present in intact nucleosomes containing a histone octamer core complexed with DNA. The antibody does not bind (or binds weakly) to free histone octamer complexes, free histones (i.e. without DNA), free DNA or free histones. Again, the antibody may be relatively unaffected by the histone PTM composition of the nucleosomes to be bound.

Therefore, in one embodiment of the invention the antibody is directed to bind to a conformational nucleosome epitope present in intact nucleosomes containing a histone octamer core complexed with DNA.

In one embodiment of the invention the antibody is directed to bind selectively to clipped nucleosomes, for example by binding to an epitope present in clipped nucleosomes wherein one or more histone tails have been removed. In this embodiment, the epitope may previously have been masked in intact nucleosomes by the presence of complete histone tails (thus preventing antibody binding). Therefore, the epitope bound by the antibody which is selective for clipped nucleosomes, may be an epitope that is not accessible in intact (i.e. whole or unclipped) nucleosomes. In one embodiment, the clipped nucleosome comprises a histone H3, H2A and/or H4 protein where the histone tail has been removed.

In one embodiment a mixture of 2 or more therapeutic antibodies may be administered to a subject. For example, without limitation, a mixture of one antibody directed to bind to a nucleosome epitope present in intact nucleosomes, together with another antibody directed to bind to a core histone epitope of histone H3.1.

The antibodies used by the inventors for immunoassay are generally mouse monoclonal antibodies.

The term “antibody” herein is used in the broadest sense and includes polyclonal and monoclonal antibodies, including intact antibodies and functional (antigen-binding) antibody fragments, including fragment antigen binding (Fab) fragments, F(ab′) 2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

As used herein, the term “binding” or “specific binding” refers to the specificity of a binder, e.g., an antibody, such that it preferentially binds to a target, such as a polypeptide antigen. When referring to a binding partner, e.g., protein, nucleic acid, antibody or other affinity capture agent, etc., “specific binding” can include a binding reaction of two or more binding partners with high affinity and/or complementarity to ensure selective hybridization under designated assay conditions. Typically, specific binding will be at least three times the standard deviation of the background signal. Thus, under designated conditions the binding partner binds to its particular target molecule and does not bind in a significant amount to other molecules present in the sample. Recognition by a binder or an antibody of a particular target in the presence of other potential interfering substances is one characteristic of such binding. Preferably, binders, antibodies or antibody fragments that are specific for or bind specifically to a target bind to the target with higher affinity than binding to other non-target substances. Also preferably, binders, antibodies or antibody fragments that are specific for or bind specifically to a target avoid binding to a significant percentage of non-target substances, e.g., non-target substances present in a testing sample. In some embodiments, binders, antibodies or antibody fragments of the present disclosure avoid binding greater than about 90% of non-target substances, although higher percentages are clearly contemplated and preferred. For example, binders, antibodies or antibody fragments of the present disclosure avoid binding about 91%, about 92%, about 93%, about 94%, about 95%, about 96%, about 97%, about 98%, about 99%, and about 99% or more of non-target substances. In other embodiments, binders, antibodies or antibody fragments of the present disclosure avoid binding greater than about 10%, 20%, 30%, 40%, 50%, 60%, or 70%, or greater than about 75%, or greater than about 80%, or greater than about 85% of non-target substances.

### Sample

The sample may be any biological fluid (or body fluid) sample taken from a subject including, without limitation, cerebrospinal fluid (CSF), whole blood, blood serum, plasma, menstrual blood, endometrial fluid, urine, saliva, or other bodily fluid (stool, tear fluid, synovial fluid, sputum), breath, e.g. as condensed breath, or an extract or purification therefrom, or dilution thereof. In a preferred embodiment, the body fluid sample is selected from blood, serum or plasma. Biological samples also include specimens from a live subject, or taken post-mortem. The samples can be prepared, for example where appropriate diluted or concentrated, and stored in the usual manner. It will be understood that methods and uses of the present invention find particular use in blood, serum or plasma samples obtained from a patient. In one embodiment, the sample is a blood or plasma sample. In a further embodiment, the sample is a serum sample. In a further embodiment both serum and plasma samples are used for the measurement of different members of an assay panel.

It will be understood that the embodiments described herein may be applied to all aspects of the invention, i.e. the embodiment described for the uses may equally apply to the claimed methods and so forth.

The invention will now be illustrated with reference to the following non-limiting examples.

### Example 1

We induced NETs formation in white cells in fresh healthy whole blood samples by addition of heparin and then demonstrated detection of the NETs material produced in plasma (Lelliott et al, International Immunology (2019) pii: dxz084). We collected whole blood samples from two healthy volunteers, in whom low levels of circulating NET material would be expected, in EDTA plasma blood collection tubes and in heparin plasma blood collection tubes. The two EDTA plasma blood collection tubes were centrifuged immediately to separate the cellular and plasma fractions to minimise contamination by large chromatin (including NET material) and the plasma was transferred to cryotubes and frozen. The two heparin plasma blood collection tubes were incubated at room temperature for 1 hour with gentle rotation of the tubes. The tubes were then centrifuged and the plasma was transferred to cryotubes and frozen.

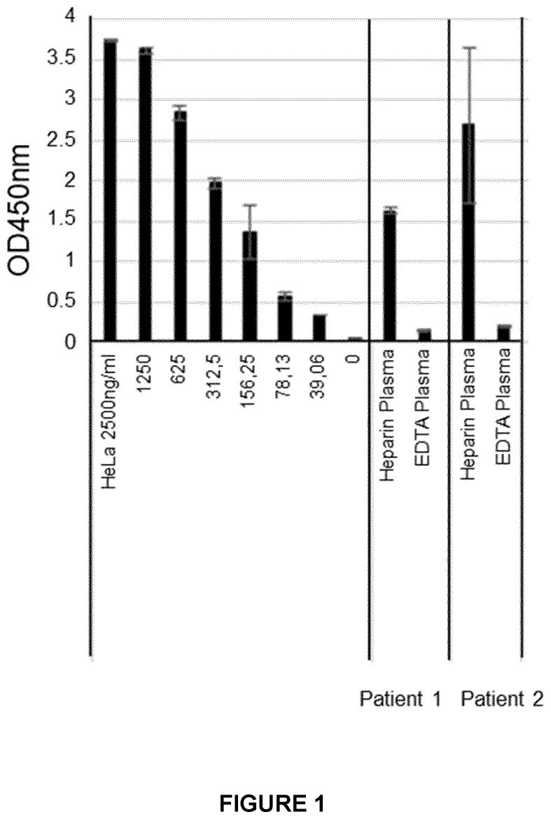

The samples were assayed for nucleosomes containing histone isoform H3.1 (H3.1-nucleosomes) using an ELISA procedure in duplicate. In brief, 20 μl of sample were added to a microtiter well containing magnetic particles precoated with an anti-histone H3.1 antibody. The sample was incubated and the magnetic particles were isolated and washed. An anti-nucleosome antibody directed to bind to a conformational nucleosome epitope conjugated to horse radish peroxidase was added to the magnetic particles. The particles were incubated and then isolated and washed. The bound anti-nucleosome antibody was measured using a coloured substrate reaction. The results are shown in FIG. 1 and show that the NET material level was high in the heparin tubes but low in the EDTA tubes. This clearly shows that elevated levels of circulating NET material can be detected in a simple low cost immunoassay test.

### Example 2

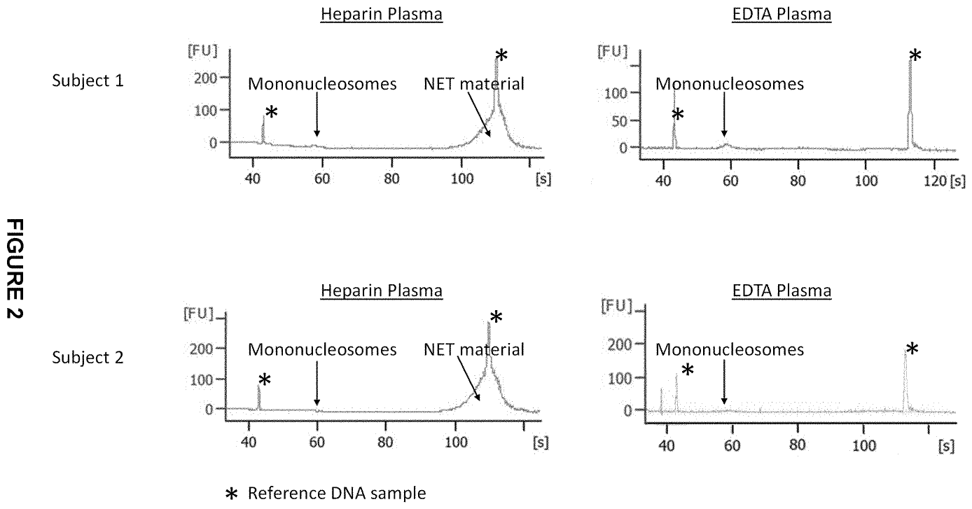

DNA was extracted from the two heparin and plasma samples described in EXAMPLE 1, and applied to a chip-based capillary electrophoresis instrument (Agilent Bioanalyzer) to analyse the DNA by fragment size. DNA fragments of approximately 150 bp size corresponding to mononucleosomes have a retention time of approximately 60 seconds. As shown in FIG. 2, the level of mononucleosome associated DNA observed was low (as expected for healthy volunteers). The DNA fragment size corresponding to NET material has a longer retention time of approximately 110 seconds. As shown in FIG. 2 the level of NET material was low in EDTA plasma (as expected for healthy volunteers), but high in the heparin plasma tubes in which NET formation was stimulated by exposure to heparin. This result confirms that the elevated nucleosome levels observed in heparin plasma in EXAMPLE 1 above were NET derived and not mononucleosomes.

### Example 3

Plasma samples were obtained from subjects diagnosed with a form of VTE and healthy subjects. The plasma samples were assayed for nucleosomes containing histone isoform H3.1 (H3.1-nucleosome) levels using an automated immunoassay instrument. Briefly, calibrant or sample is incubated with an acridinium ester labelled anti-nucleosome antibody and assay buffer for 1800 seconds at 37° C. Magnetic beads coated with an anti-histone H3.1 antibody were added and the mixture was incubated a further 900 seconds. The magnetic beads were then isolated, washed 3 times and magnetic bound acridinium ester was determined by luminescence output over 7000 milliseconds. Elevated levels are observed in the VTE samples compared to healthy subjects.

### Example 4

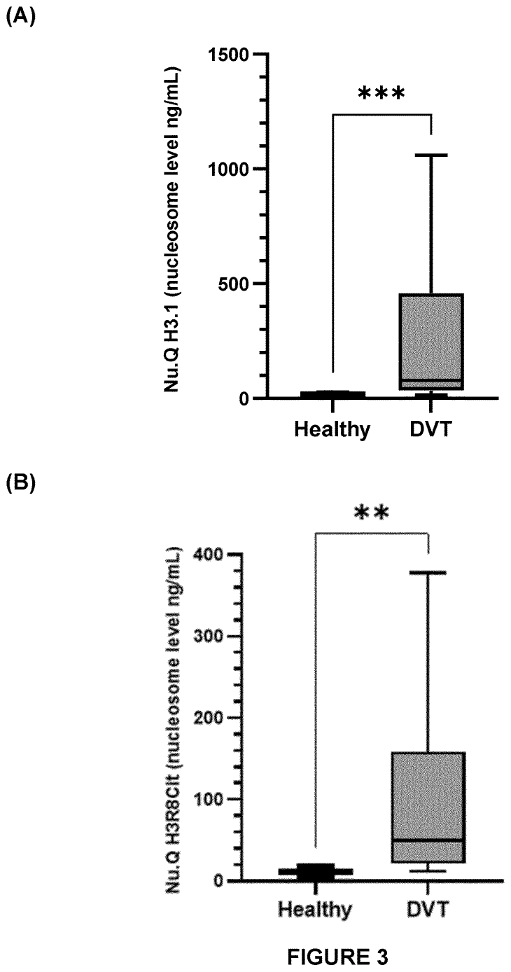

Plasma samples were obtained from subjects diagnosed with DVT, some diagnosed with DVT and PE and healthy subjects. The plasma samples were assayed for H3.1-nucleosome levels as described in EXAMPLE 3. The results are shown in FIG. 3A. Elevated levels are observed in the DVT samples compared to healthy subjects. Interestingly, the plasma sample from one subject which had received no treatment for the main diagnosis (an untreated subject), showed the highest H3.1-nucleosome concentration.

The same samples were assayed for nucleosomes containing citrullinated histone H3 (H3R8Cit). The assay was the same as described in EXAMPLE 3 for H3.1-nucleosomes, although an anti-histone H3R8Cit antibody is used instead of an anti-histone H3.1 antibody. The results are shown in FIG. 3B. Elevated levels are observed in the DVT samples compared to healthy subjects.

1. A method of detecting a subject in need of medical treatment for a disorder of hemostasis or thrombosis comprising:
(i) contacting a body fluid sample obtained from the subject with a binding agent to detect or measure the level of cell free nucleosomes or a component thereof; and
(ii) using the level of cell free nucleosomes as an indicator that the subject is in need of medical treatment for a disorder of hemostasis or thrombosis.

2. A method for diagnosing the presence or the risk of development of a disorder of hemostasis or thrombosis or of monitoring the progress of a disorder of hemostasis or thrombosis in a subject suffering from the condition, comprising:
(i) contacting a body fluid sample obtained from the subject with a binding agent to detect or measure the level of cell free nucleosomes or a component thereof;
(ii) repeating step (i) on one or more occasions; and
(iii) using any changes in the level of cell free nucleosomes or component thereof to monitor the progression of the disorder of hemostasis or thrombosis in the subject.

3. A method of assigning a risk of an adverse outcome to a subject suffering from a disorder of hemostasis or thrombosis comprising:
(i) contacting a body fluid sample obtained from the subject with a binding agent to detect or measure the level of cell free nucleosomes or a component thereof; and
(ii) using the level of cell free nucleosomes detected to assign the likelihood of an adverse outcome to said subject,
wherein a subject identified with a high likelihood of an adverse outcome is assigned for medical intervention.

4. The method of claim 1, wherein the disorder of hemostasis or thrombosis is selected from venous thromboembolism (VTE), vasculitis, disseminated intravascular coagulation (DIC) or antiphospholipid syndrome (APS).

5. The method of claim 4, wherein the VTE is deep vein thrombosis (DVT) or pulmonary embolism (PE).

6. The method of claim 4, wherein the vasculitis is eosinophilic granulomatosis with polyangiitis (Churg-Strauss syndrome), temporal arteritis (giant cell arteritis), granulomatosis with polyangiitis (Wegener's granulomatosis), Henoch-Schoenlein purpura, Kawasaki condition, microscopic polyangiitis, polyarteritis nodosa, polymyalgia rheumatica, Takayasu arteritis, Behcet's condition, Buerger's condition, Cogan's syndrome, Cryoglobulin-associated vasculitis, hypersensitivity vasculitis, primary angiitis of the central nervous system or rheumatoid vasculitis.

7. The method of claim 4, wherein the DIC is associated with trauma, organ destruction, sepsis or infection (including coronavirus disease 2019 [COVID-19] infection), obstetric disorders, malignancies, vascular disorders, and toxic or immunological reactions.

8. The method of claim 4, wherein the APS is associated with thrombosis.

9. The method of claim 1, wherein the body fluid sample is a blood, serum or plasma sample.

10. (canceled)

11. The method of claim 1, wherein the component of the cell free nucleosome comprises an epigenetic feature of the cell free nucleosome.

12. The method of claim 11, wherein the epigenetic feature is a histone isoform, such as a histone isoform of a core nucleosome.

13. The method of claim 12, wherein the histone isoform is H3.1.

14. The method of claim 12, wherein the epigenetic feature is a histone post translational modification (PTM).

15. (canceled)

16. The method of claim 1, wherein the level of cell free nucleosomes or component thereof is detected or measured using an immunoassay, immunochemical, mass spectroscopy, chromatographic, chromatin immunoprecipitation or biosensor method.

17. (canceled)

18. The method of claim 1, wherein the method of detection or measurement comprises: (i) contacting the sample with a first binding agent which binds to an epigenetic feature of a cell free nucleosome; (ii) contacting the sample bound by the first binding agent in step (i) with a second binding agent which binds to cell free nucleosomes; and (iii) detecting or quantifying the binding of the second binding agent in the sample.

19. The method of claim 1, wherein the subject is a human or an animal subject.

20-23. (canceled)

24. The method of claim 1, wherein the level of cell free nucleosomes is detected or measured as one of a panel of measurements.

25-27. (canceled)

28. The method of claim 24, wherein the panel comprises one or more of a test to measure activated partial thromboplastin time (aPTT), a LA sensitive aPTT (LA-aPTT) test, a diluted or modified Russell viper venom screen (dRVVT, MRVVT), a prothrombin time (PT) test, an assay for anticardiolipin antibody, and/or an assay for anti-beta2-glycoprotein antibody.

29. (canceled)

30. The method of claim 1, wherein the method is carried out in combination with a Wells score or a PERC Rule.

31. The method of claim 1, wherein the method is carried out in combination with an imaging technique such as an ultrasound scan, ventilation-perfusion (VQ) scan or computed tomography (CT) pulmonary angiogram.

32. (canceled)


SEQUENCE LISTING (1 sequence)

SEQ ID NO: 1 - protein, 10 aa, Homo sapiens
KSAPATGGVK